(19) United States
(12) Patent Application Publication
(10) Pub. No.: US 2007/0268996 A1
(43) Pub. Date: Nov. 22, 2007

(54) RADIATION IMAGING APPARATUS AND RADIATION IMAGING METHOD

(76) Inventors: Akira Hagiwara, Tokyo (JP); Masatake Nukui, Tokyo (JP); Akihiko Nishide, Tokyo (JP); Makoto Gohno, Tokyo (JP)

Correspondence Address: GENERAL ELECTRIC CO.;GLOBAL PATENT OPERATION, 187 Danbury Road, Suite 204, Wilton, CT, 06897-4122, US

(21) Appl. No.: 11/745,597

(22) Filed: Jul. 26, 2007

(30) Foreign Application Priority Data
May 8, 2006 (JP) 2006-129391

(51) Int. Cl. H05G 1/60; A61B 6/00; G01N 23/00; G21K 1/12

(52) U.S. Cl. 378/4

(57) ABSTRACT

Embodiments of the present invention improve image quality by preventing artifacts from developing. From the projection data obtained by performing a scan for the imaging area of a subject, a tomographic image for a cross-sectional plane of the imaging area is reconstructed. Based on the tomographic image, the density distribution of the imaging area of the subject is then calculated. Thereafter, the scattered radiation beam data is calculated depending on the density distribution, and the projection data is corrected using the scattered radiation beam data. Finally the corrected image is reconstructed based on the corrected projection data.

### BACKGROUND OF THE INVENTION

1. Field of the Invention

The field of the present invention generally relates a radiation imaging apparatus and a radiation imaging methods. In particular the field of the present invention relates to a radiation imaging apparatus and a radiation imaging method, which allow reconstructing a scattered radiation corrected image to which scattered radiation correction processing is applied.

2. Description of Related Art

A radiation imaging apparatus including X-ray CT (computed tomography) apparatus emits radiation such as X-ray into an imaging area of a subject, and then performs a scan for detecting the radiation transmitted through the imaging area of the subject in order to obtain the projection data. The tomographic image with respect to the cross-sectional plane of the imaging area may be reconstructed based on the projection data obtained from the scan performed. Such a radiation imaging apparatus has been widely used in vast fields such as medical use and industrial use.

When imaging a subject, more specifically, the X-ray CT apparatus moves its X-ray tube and multi-column X-ray detector so as to rotate in the periphery of the subject around the body axis direction of the subject and then performs a scan. Here, the X-ray tube disperse an X-ray beam radially, or in the form of cone beam, in the channel direction along with the rotating direction of the detector rotating around the subject, and in the row direction along with the rotating axis of the rotation, and the multi-column X-ray detector, having a plurality of detecting elements arrayed along with the channel direction and row direction, detects the X-ray beam transmitted through the subject, thereby a scan is performed. Such a scan may be performed as an axial scan, a helical scan, and the like.

Then, a series of tomographic images serially arranged in a plurality of axial plane in the body axis direction of the subject may be reconstructed based on the projection data obtained from the scan performed. At this point, for example, according to an image reconstruction method based on such method as Feldkamp method, including image reconstruction methods referred to as the three-dimensional back projection method and the cone beam back projection method, a weighted addition processing on the projection data that each opposes another, a tomographic image is restructured corresponding to the axial plane which is the vertical plane with the body axis direction defined as vertical line.

When imaging a subject using an X-ray CT apparatus, part of the X-ray transmitted from the X-ray tube to the imaging area of the subject at the time of scanning may be scattered as scattered radiation beam in the scattering direction, which is different from the radiation direction from the X-ray tube to each of the detecting elements in the multi-column X-ray detector, due to the imaging area of the subject therebetween. This phenomenon causes the projection data to include the scattered radiation beam data as noise component. Therefore, the tomographic image that is reconstructed based on the projection data may have some artifacts because of the influence of the scattered radiation beam, resulting in some degradation of the image quality.

To suppress the occurrence of such inconvenience, a collimator may be used for shielding the scattered radiation beam between each detecting element in the multi-column X-ray detector to prevent the radiation of the scattered radiation beam from reaching to the detecting elements (see for example patent reference 1).

In addition, to suppress the occurrence of such inconvenience, the scattered radiation beam data included as noises in the projection data obtained from the scan, can be calculated by the computation, and then, the scattered radiation correction processing may be performed by using the scattered radiation beam data. For example, the scattered radiation beam data determined may be used to correct the projection data, and a tomographic image may be reconstructed based on the corrected projection data to obtain the scattered corrected image with the scattered radiation beam corrected (see for example patent reference 2 and 3).

[Patent Reference 1]

### Japanese Unexamined Patent Publication No. 2005-87618

[Patent Reference 2]

### Japanese Unexamined Patent Publication No. 2000-197628

[patent reference 3] Japanese Unexamined Patent Publication No. H7(1995) -213517

When performing the scattered radiation correction processing as stated above, the characteristics of a scattered radiation beam that the X-ray transmitted from the X-ray tube is scattered at the subject due to phenomenon for example such as the photoelectric effect, Rayleigh scattering, and Compton scattering can be calculated by the computation corresponding to the energy distribution of the radiated X-ray in order to determine the scattered radiation beam data included in the projection data. Then the scattered radiation beam data thus determined may be used to correct for the projection data. Alternatively, there may be a case in which the scattered radiation beam data is calculated so as to accommodate with the transmission length that the radiation transmits in the subject, in order to perform a much precise correction on the projection data. In this manner a highly precise scattered radiation correction is accomplished.

However, there are cases where the inconvenience as stated above could not be ameliorated well even with the correction schemes as above.

More specifically, when there are high- and low-density areas included in the imaging area of the subject, the behavior of the scattered radiation beam may vary in accordance with the density profile. However, since the correction is based on the assumption that the density is uniform in the imaging area of the subject, the correction of projection data may or may not be sufficiently performed so as not to include the scattered radiation beam data as noise, so that the tomographic image reconstructed by using this projection data may have some artifacts, resulting in the degradation of image quality.

In particular, the number of detecting elements in the multi-column X-ray detector device is increasing in the row direction these days to allow acquisition of projection data from a wider area. This causes significant occurrence of artifacts on the image, and sometimes the inconvenience that degrades the image quality may become apparent. For example, when imaging an area including the liver in the subject as the imaging area, there are the area including the liver and the area without the liver in the row direction. Since the density of the area differs in both imaging area, the behavior of the scattered radiation beam vary respectively, so that the noise caused by the scattered radiation beam may not be sufficient eliminated from the projection data. This involves the significant shading occurred on the image, resulting in the degradation of image quality.

Therefore, a need exists to provide a radiation imaging apparatus and radiation imaging method that overcome the above-noted deficiencies and improve image quality.

### SUMMARY OF THE INVENTION

In an aspect of the present invention, a radiation imaging apparatus includes a scan unit. The scan unit may include a radiation section configured to emit a radiation beam and a detecting section having a plurality of detecting elements arranged to detect the radiation emitted from the radiation section. The scan unit may be configured to perform a scan in which the radiation section emits the radiation beam to an imaging area of the subject, and the detecting section detects the radiation beam transmitted through the imaging area to obtain projection data of the imaging area. The radiation imaging apparatus may further include a scan condition setting unit configured to set a scan condition for a scan performed by the scan unit. The radiation imaging apparatus may further include a scattered radiation beam data calculation unit, configured to calculate scattered radiation beam data. The scattered radiation beam data may be calculated by estimating scattered radiation beam scattered in a scattering direction in the radiation beam emitted from the radiation section to the imaging area of the subject in accordance with a scan condition set by the scan condition setting unit. The scattering direction direction may be different from a radiation direction in which the radiation beam is emitted from the radiation section to each of the detecting elements, and the radiation imaging apparatus may further include an image reconstruction unit configured to reconstruct a scattered radiation corrected image to which scattered radiation correction processing is applied with respect to a cross-sectional plane in the imaging area of the subject and the scattered radiation beam data calculated by the scattered radiation beam data calculation unit. The scattered radiation correction processing may use projection data obtained from a scan performed by the scan unit in accordance with a scan condition set by the scan condition setting unit. Additionally, the image reconstruction unit may be configured to reconstruct a tomographic image with respect to a cross-sectional plane of the imaging area based on the projection data. The scattered radiation beam data calculation unit may include a density distribution calculation unit for configured to calculate a density distribution of the imaging area, based on the tomographic image reconstructed by the image reconstruction unit, and to calculate the scattered radiation beam data based on the density distribution calculated by the density distribution calculation unit.

In another aspect of the present invention, a radiation imaging method may be provided. The radiation imaging method may permit a scan unit to perform a scan in accordance with a scan condition. The scan unit may include a radiation section configured to emit a radiation beam and a detecting section having a plurality of detecting elements arranged to detect the radiation beam emitted from the radiation section. The radiation section may be configured to emit the radiation beam to an imaging area to obtain projection data of the imaging area. The radiation imaging method include a scattered radiation beam data calculating step of calculating scattered radiation beam data by estimating a scattered radiation beam scattered in a scattering direction which is different from a radiation direction in which a radiation beam is emitted from the radiation section to each of detecting elements in the detecting section to the imaging area of the subject in accordance with the scan condition. The radiation imaging method may include a scattered radiation corrected image reconstructing step of reconstructing a scattered radiation corrected image, and of applying scattered radiation correction processing to the scattered radiation corrected image with respect to a cross-sectional plane in the imaging area of the subject, by using the projection data obtained from a scan performed by said scan unit and the scattered radiation beam data calculated in said scattered radiation beam data calculating step. The scattered radiation beam data calculating step may include a tomographic image reconstructing step of reconstructing a tomographic image with respect to a cross-sectional plane of the imaging area based on the projection data. THe radiation imaging method may include a density distribution calculating step of calculating a density distribution of the imaging area, based on the tomographic image reconstructed in the tomographic image reconstructing step, and a data calculating step of calculating the scattered radiation beam data based on the density distribution calculated in the density distribution calculating step.

According to embodiments of the present invention, a radiation imaging apparatus and radiation imaging method may be provided which can improve the image quality.

### BRIEF DESCRIPTION OF THE DRAWINGS

FIG. 1 shows a schematic block diagram illustrating the overview of the X-ray CT apparatus 1 in accordance with a first preferred embodiment of the present invention;

FIG. 2 shows a schematic diagram illustrating some important members of the X-ray CT apparatus 1 in accordance with the first preferred embodiment of the present invention;

FIG. 3 shows a schematic block diagram illustrating the arrangement of the central processing unit 30 in accordance with the first preferred embodiment of the present invention;

FIG. 4 shows a schematic perspective view illustrating the arrangement of the object transporter unit 4 in accordance with the first preferred embodiment of the present invention;

FIG. 5 shows a schematic flow diagram illustrating the operation of the X-ray CT apparatus 1 in accordance with the first preferred embodiment of the present invention;

FIG. 6 shows a schematic side elevation illustrating the behavior of scattered radiation that scatters due to the imaging area of the subject at the time of performing a scan of the imaging area of the subject by means of the scanning gantry 2, in accordance with the first preferred embodiment of the present invention;

FIG. 7 shows a schematic block diagram illustrating the arrangement of the central processing unit 30 in accordance with a second preferred embodiment of the present invention;

FIG. 8 shows a schematic flow diagram illustrating the operation of the X-ray CT apparatus 1 in accordance with the second preferred embodiment of the present invention; and

FIG. 9 shows a schematic flow diagram illustrating the operation of the X-ray CT apparatus 1 in accordance with a third preferred embodiment of the present invention.

### DETAILED DESCRIPTION OF THE INVENTION

Some best modes for carrying out the present invention may be described in greater detail herein below. Embodiments of the invention, however, are not limited to these best modes.

### First Embodiment

From now on a first preferred embodiment of the present invention may be described in greater detail.

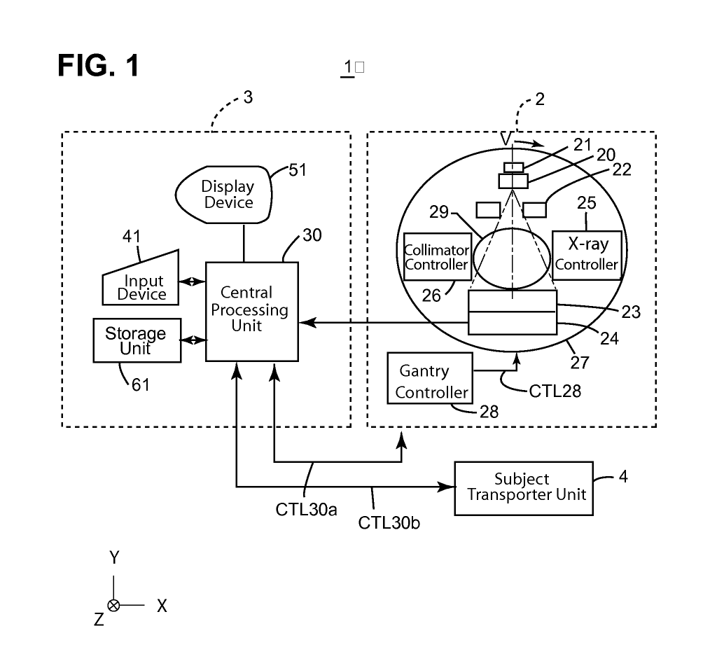

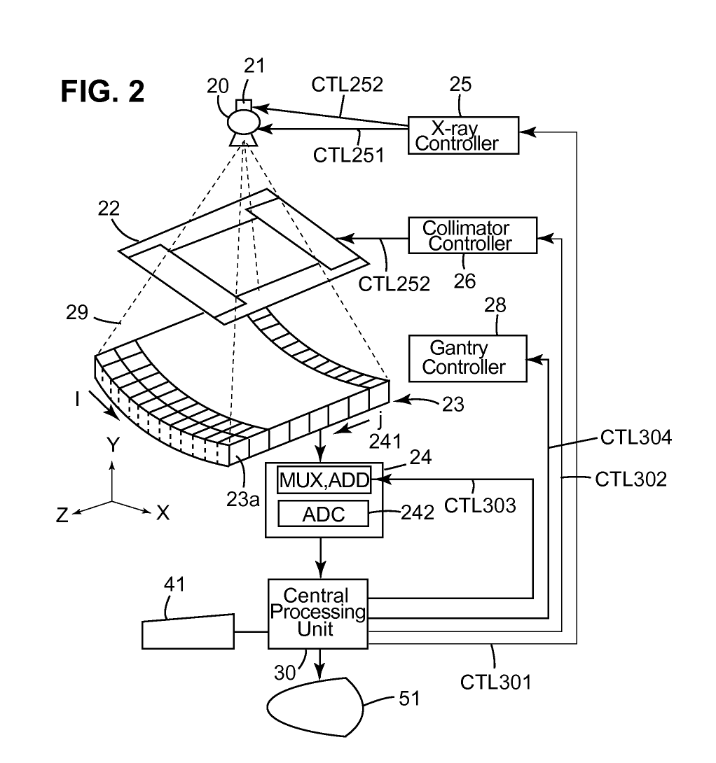

Now referring to FIG. 1, there is shown a schematic block diagram indicating the overview of an X-ray CT apparatus 1, in accordance with the first preferred embodiment of the present invention. Referring to FIG. 2, there is shown a perspective view of some important part of the X-ray CT apparatus 1 in accordance with a first preferred embodiment of the present invention.

As shown in FIG. 1, the X-ray CT apparatus 1 has a scanning gantry 2, an operating console 3, and a subject transporter unit 4. The X-ray CT apparatus 1 uses the projection data obtained by performing a scan by emitting X-ray to the subject then detecting the X-ray transmitted through the subject to reconstruct image for the subject.

Now the scanning gantry 2 may be described in greater detail.

The scanning gantry 2, as shown in FIG. 1, has an X-ray tube 20, an X-ray tube transporter unit 21, a collimator 22, an X-ray detector 23, a data acquisition system 24, an X-ray controller 25, a collimator controller 26, a revolving unit 27, and a gantry controller 28. The scanning gantry 2 will obtain the projection data for the imaging area of the subject from a scan performed by emitting X-ray from the X-ray tube 20 to the imaging area of the subject and then by detecting the X-ray transmitted through the imaging area by the X-ray detector 23. In this preferred embodiment the subject transported into an imaging space 29 by the subject transporter unit 4 is scanned with X-ray to obtain the projection data of the subject, based on the control signal CTL 30a from the operating console 3 in accordance with the scan condition set by a scan condition setting unit 302 of the operating console 3 as may be described later.

More specifically, in the scanning gantry 2, as shown in FIG. 2, the X-ray tube 20 and the X-ray detector 23 are opposingly arranged on both side of the imaging space 29 into which the subject may be carried. The collimator 22 is placed between the X-ray tube 20 and the X-ray detector 23, for shaping the X-ray emitted to the subject within the imaging space 29 from the X-ray tube 20. The scanning gantry 2 revolves or rotates the X-ray tube 20, the collimator 22, and the X-ray detector 23 around the subject to performing a scan, namely emitting X-ray from the X-ray tube 20 at each given view angle v around the subject, and detecting the X-ray transmitted through the subject with the X-ray detector 23 in order to obtain the projection data of the imaging area of the subject. Here, as shown in FIG. 1, the view angle v is an angle at which the X-ray tube 20 has revolved from the y direction that is a vertical direction set as 0 degree. Next the parts of the scanning gantry 2 may be described one by one.

The X-ray tube 20, which may be a rotary anode type for example, emits X-ray to the subject. The X-ray tube 20, as shown in FIG. 2, emits the X-ray of a given intensity to the imaging area of the subject through the collimator 22 based on the control signal CTL 251 sent from the X-ray controller 25. Then the X-ray tube 20 revolves around the subject by the revolving unit 27 on the center of the body axis direction z, which is along with the moving direction that the subject transporter unit 4 carries the subject into the imaging space 29, so as to emit X-ray from the round of the subject. The X-ray tube 20 emits X-ray so as to diverge radially in the channel direction i, and in the row direction j. Here, the channel direction i is the rotating direction that the X-ray tube 20 is revolved by the revolving unit 27, and the row direction j is the rotating axis direction of the revolving. The X-ray emitted from the X-ray tube 20 is shaped by the collimator 22 to a cone shaped beam, and is radiated toward the X-ray detector 23.

The X-ray tube transporter unit 21, as shown in FIG. 2, moves the X-ray tube 20 so that the center of the X-ray tube 20 moves in the row direction j, based on the control signal CTL 252 from the X-ray controller 25.

The collimator 22, as shown in FIG. 2, is arranged between the X-ray tube 20 and the X-ray detector 23. The collimator 22 for example includes shields for blocking X-ray and preventing X-ray from penetrating, and there are two shields respectively in both channel direction and row direction j. The collimator 22 moves independently two shields arranged respectively in the channel direction i and the row direction j based on the control signal CTL 261 from the collimator controller 26, to shield the X-ray emitted from the X-ray tube 20 in the respective directions to shape the beam in a cone shaped beam to adjust the radiation area of X-ray to be emitted to the subject. In other words, the collimator 22 adjusts the size of the aperture through which the radiated X-ray from the X-ray tube 20 transmits to the subject by moving the shields in the channel direction i to adjust the radiation angle of X-ray to a predetermined fan angle, while on the other hand varies the size of the aperture by moving the shields in the row direction j to adjust the radiation angle of X-ray to a predetermined cone angle.

The X-ray detector 23 detects the X-ray emitted from the X-ray tube 20 and transmitted through the subject placed in the imaging space 29 to obtain the projection data of the subject. The X-ray detector 23 also revolves around the subject by the revolving unit 27, along with the X-ray tube 20. Then the X-ray detector 23 detects the X-ray emitted from the X-ray tube 20 and transmitted through the subject from the round of the subject to generate the projection data.

As shown in FIG. 2, the X-ray detector 23 has a plurality of detecting elements 23a for detecting X-ray emitted from the X-ray tube 20. The X-ray detector 23 is the type so-called multi-column X-ray detector, which has for example two-dimensionally arrayed detecting elements 23a arranged in the channel direction i along the revolving direction that the X-ray tube 20 rotates around the subject in the imaging space 29 by the revolving unit 27, and in the row direction j along the revolving axis direction which is the center axis at the time of rotating the X-ray tube 20 by the revolving unit 27. For example the X-ray detector 23 may have about 1000 detecting elements 23a in the channel direction i, and about 8 detecting elements 23a in the row direction j. The X-ray detector 23 forms a concaved detection plane by a plurality of detecting elements 23a two-dimensionally arrayed.

The detecting elements 23a which forms the X-ray detector 23 may be made of any type of solid detector, and include a scintillator (not shown in the figure) for transducing X-ray to light, and a photodiode (not shown in the figure) for transducing the light transduced by the scintillator to electric charge. Here, it should be noted that the X-ray detecting elements 23a may not be limited thereto, and may also be made as a semiconductor detecting element using the Cadmium-Tellurium (Cd—Te), or as a ion chamber type detecting element using the Xenon (Xe) gas. There is also a collimator (not shown in the figure) for preventing the scattered X-ray from penetrating to the detecting elements 23a in the channel direction i of the X-ray detector 23.

The data acquisition system 24 is provided for acquiring the projection data from the X-ray detector 23. The data acquisition system 24 acquires the projection data made from the detected X-ray by the detecting elements 23a of the X-ray detector 23, and outputs the acquired projection data to the operating console 3. As shown in FIG. 2, the data acquisition system 24 has a multiplexer-adder selector (MUX, ADD) circuit 241 and A/D converter (ADC) 242. The multiplexer-adder selector circuit 241 selects the acquired projection data from the detecting elements 23a of the X-ray detector 23 in accordance with the control signal CTL 303 from a central processing unit 30, or adds by changing the combination thereof, and outputs the result to the A/D converter 242. The A/D converter 242 converts the projection data, which is selected or added as a given combination by the multiplexer-adder selector circuit 241, from the analog signal into the digital signal, and outputs the converted projection data to the central processing unit 30, and then, the data is stored in the storage unit 61.

The X-ray controller 25, as shown in FIG. 2, outputs the control signal CTL 251 to the X-ray tube 20 based on the control signal CTL 301 from the central processing unit 30, to control the emission of X-ray. The X-ray controller 25 for example controls the tube current and emission time of the X-ray tube 20. The X-ray controller 25 also outputs the control signal CTL 252 to the X-ray tube transporter unit 221 based on the control signal CTL 301 from the central processing unit 30 to control so as to move the radiation center of the X-ray tube 20 in the row direction j.

The collimator controller 26, as shown in FIG. 2, outputs the control signal CTL 261 to the collimator 22 based on the control signal CTL 302 from the central processing unit 30, to control the collimator 22 so as to shape the X-ray emitted from the X-ray tube 20 to the subject.

The revolving unit 27, as shown in FIG. 1, is in the form of a cylinder, and has the imaging space 29 formed in the center part. The revolving unit 27 drives a motor (not shown in the figure) for example, based on the control signal CTL 28 from the gantry controller 28 to rotate around the body axis direction z of the subject within the imaging space 29. In other words, the revolving unit 27 rotates in the channel direction i around the row direction jas the rotating axis. The revolving unit 27 equips the X-ray tube 20, the X-ray tube transporter unit 21, the collimator 22, the X-ray detector 23, the data acquisition system 24, the X-ray controller 25, and the collimator controller 26, and supports these units. The revolving unit 27 supplies electricity to these units through a slip ring (not shown in the figure). The revolving unit 27 revolves these units around the subject to change relative position, in the revolving direction, between these units and the subject carried into the imaging space 29.

The gantry controller 28, as shown in FIG. 1 and FIG. 2, outputs the control signal CTL 28 to the revolving unit 27 based on the control signal CTL 304 from the central processing unit 30 of the operating console 3, to control the rotation of the revolving unit 27.

Now the operating console 3 may be described in greater detail.

The operating console 3, as shown in FIG. 1, has the central processing unit 30, an input device 41, a display device 51, and a storage unit 61.

The central processing unit 30 in the operating console 3 performs a variety of processing based on the instruction input to the input device 41 by the operator. The central processing unit 30 may include a computer, and a program for functioning the computer as a variety of means.

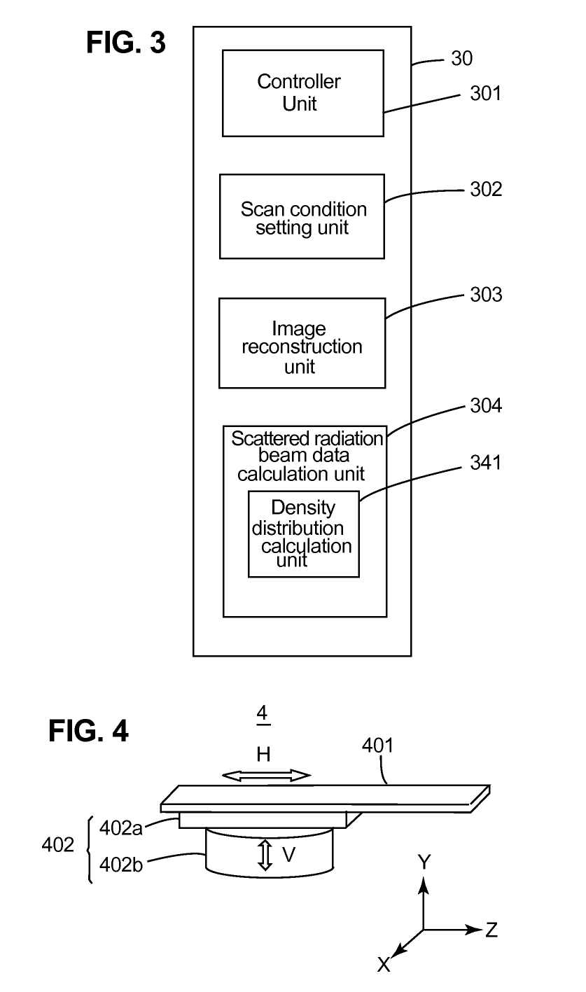

Now referring to FIG. 3, there is shown a schematic block diagram indicating the arrangement of the central processing unit 30 in accordance with the first preferred embodiment of the present invention.

The central processing unit 30, as shown in FIG. 3, has a controller unit 301, the scan condition setting unit 302, an image reconstruction unit 303, and a scattered radiation data calculation unit 304. Each part contains a program for functioning the computer as a variety of means.

The controller unit 301 is provided for controlling parts of the X-ray CT apparatus 1. The controller unit 301 controls the units based on the instruction input to the input device 41 by the operator. For example, the controller unit 301 controls each part to perform a scan in accordance with the scan condition set by the scan condition setting unit 302 based on the instruction input to the input device 41 by the operator. More specifically, the controller unit 301 outputs the control signal CTL 30b into the subject transporter unit 4, to cause the subject transporter unit 4 to carry in and move the subject to the imaging space 29. The controller unit 301 outputs the control signal CTL 304 into the gantry controller 28, to cause the revolving unit 27 of the scanning gantry 2 to revolve. The controller unit 301 outputs the control signal CTL 301 into the X-ray controller 25, to cause the X-ray tube 20 to emit X-ray. The controller unit 301 outputs the control signal CTL 302 into the collimator controller 26, to control the collimator 22 to shape the X-ray. The controller unit 301 also outputs the control signal CTL 303 into the data acquisition system 24, to control to acquire the projection data obtained by the detecting elements 23a of the X-ray detector 23.

The scan condition setting unit 302 sets a scan condition for operating each part when performing a scan, based on the scan parameter input to the input device 41 by the operator. For example, the scan condition setting unit 302 sets a scan condition including slice thickness, scan start position, scan end position, scan pitch, X-ray beam width, tube current value, tube voltage value, etc. The scan condition setting unit 302 then outputs the scan condition data to the controller unit 301 to control each part.

The image reconstruction unit 303 reconstructs a tomographic image of a cross-section of the subject, as a digital image made of a plurality of pixels, based on the projection data acquired by the data acquisition system 24 after performing a scan. For example, the image reconstruction unit 303 reconstructs images of a plurality of cross-sections of the subject from the projection data obtained by performing the scan, using the CT value as pixel value. For example, it performs image reconstruction by means of the cone beam back projection method. Thus the image reconstruction unit 303 uses a plurality of projection data sets, corresponding to the pixels on the image reconstruction plane, to reconstruct images of the cross-sections of the subject. In the preferred embodiment the image reconstruction unit 303 will perform a preprocessing including such as the offset correction, logarithm correction, X-ray dose correction, sensitivity correction, etc., to the projection data acquired by the data acquisition system 24. The image reconstruction unit 303 then performs a filtering processing onto the preprocessed projection data. In this preferred embodiment the image reconstruction unit 303 performs a filtering including a Fourier Transform, a convolution of image reconstruction function, and then an invert Fourier Transform. Thereafter, a three-dimensional back projection processing is applied to the projection data thus filtered, and then, a postprocessing is applied thereto to generate the image data.

In the preferred embodiment, the image reconstruction unit 303 uses the projection data obtained by performing a scan with the scanning gantry 2 so as to accommodate to the scan condition set by the scan condition setting unit 302, and the scattered radiation beam data calculated by the scattered radiation beam data calculation unit 304 to reconstruct the scattered radiation corrected image having the scattered radiation corrected with respect to the cross-sectional planes of the imaging area of the subject.

Although details may be described later, when reconstructing the scattered radiation corrected image, the image reconstruction unit 303 receives, from the storage unit 61, the projection data obtained by performing a scan with the scanning gantry 2 in accordance with the scan condition set by the scan condition setting unit 302, then it uses the projection data to reconstruct a tomographic image for the cross-sectional plane of the imaging area of the subject. Then, the tomographic image thus reconstructed may be used to calculates the density distribution in the imaging area of the subject by the scattered radiation beam data calculation unit 304, the scattered radiation beam data is derived from the density distribution, and the image reconstruction unit 303 receives from the storage unit 61 once again the projection data obtained by performing a scan, to perform the scattered radiation correction for the projection data by using the scattered radiation beam data calculated by the scattered radiation beam data calculation unit 304. Thereafter, based on the projection data having scattered radiation correction processing applied, the image reconstruction unit 303 will reconstruct an image having the scattered radiation beam corrected.

For the X-ray emitted by the X-ray tube 21 to the imaging area of the subject in accordance with the scan condition set by the scan condition setting unit 302, the scattered radiation data calculation unit 304 calculates the scattered radiation beam data, by estimating the scattered radiation beam scattered by the imaging area of the subject in the scattering direction which is different from the radiation direction in which the X-ray is emitted from the X-ray tube 21 to each of the detecting elements 23a of the X-ray detector 23.

As shown in FIG. 3, the scattered radiation beam data calculation unit 304 includes a density distribution calculation unit 341. In the present embodiment, the density distribution calculation unit 341 uses the tomographic image reconstructed by the image reconstruction unit 303 to calculate the distribution density in the imaging area of the subject. Then the scattered radiation beam data calculation unit 304 uses the density distribution thus calculated by the density distribution calculation unit 341 to calculate the scattered radiation beam data. The scattered radiation beam data calculation unit 304 calculates the scattered radiation beam data by estimating the scattered radiation beam corresponding to the scan condition, set by the scan condition setting unit 302, and to the density distribution, calculated by the density distribution calculation unit 341, based on the distribution radiation beam characteristics information stored in the storage unit 61 as may be described later.

The input device 41 of the operating console 3 may include, for example, a keyboard and a mouse. The input device 41, in response to the input operation of the operator, inputs to the central processing unit 30 various information and instructions such as the scan parameters and subject information. For example, when setting the actual scan condition, the input device 41 inputs, as scan parameters, data of the scan start position, the scan end position, the scan pitch, the X-ray beam width, the tube current value, and the slice thickness, in accordance with the instruction from the operator.

The display device 51 of the operating console 3 includes a CRT, which displays one or more image(s) on the display screen based on the instruction from the central processing unit 30. In the present embodiment, the display device 51 displays, for example, a scattered radiation corrected image, reconstructed by the image reconstruction unit 303, on the display screen.

The storage unit 61 of the operating console 3 is composed of memory devices for storing various data. The storage unit 61 is accessed by the central processing unit 30 to obtain the stored data when required. In the present preferred embodiment, the storage unit 61 stores the scattered radiation beam characteristics information in which characteristics of the scattered radiation beam are associated with the scan condition and the density in the imaging area of the subject.

Now the subject transporter unit 4 may be described in greater detail.

The subject transporter unit 4 carries in and out the subject to and from the imaging space 29.

Now referring to FIG. 4, there is shown a perspective view illustrating the arrangement of the subject transporter unit 4 in accordance with the first preferred embodiment of the present invention.

As shown in FIG. 4, the subject transporter unit 4 has a table 401 and a table transporter unit 402.

The table 401 of the subject transporter unit 4 is formed such that the subject stage plane on which the subject is mounted is along the horizontal plane, so that the stage supports the subject. For example, the subject will lie down on his/her back on the table and is supported by the table 401 of the subject transporter unit 4.

The table transporter unit 402 of the subject transporter unit 4 has a horizontal transporter unit 402a for moving the table 401 in the horizontal direction H along the body axis z of the subject, and a vertical transporter unit 402b for moving the table 401 of the subject transporter unit 4 in the vertical direction V perpendicular to the horizontal direction H. The table transporter unit 402 moves the table 401 to carry the subject into the inside of the imaging space 29 based on the control signal CTL 30b from the central processing unit 30.

Now the operation of the X-ray CT apparatus 1 in accordance with the preferred embodiment of the present invention may be described in greater detail herein below.

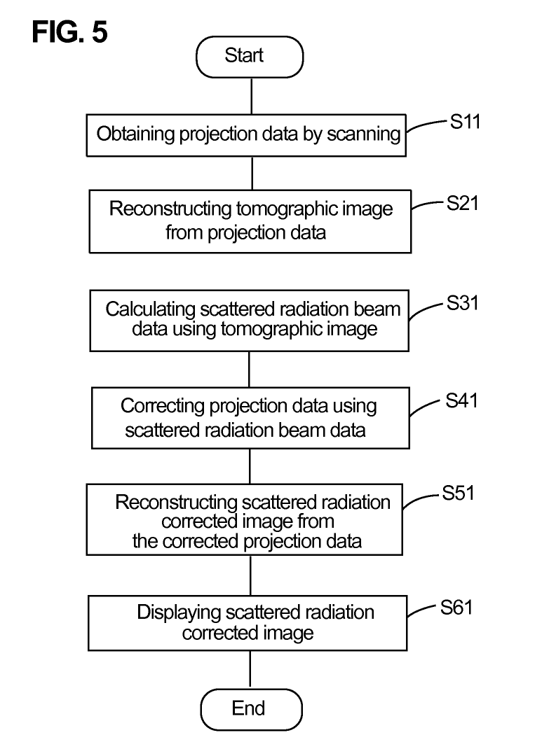

Now referring to FIG. 5, there is shown a schematic flow diagram illustrating the operation of the X-ray CT apparatus 1 in accordance with the first preferred embodiment of the present invention.

First as shown in FIG. 5, a scan is performed to obtain projection data (S11).

Here the scanning gantry 2 scans with X-ray the imaging area of the subject that is moved into the imaging space 29 by the subject transporter unit 4 in accordance with the scan condition set by the scan condition setting unit 302 to obtain the projection data of the imaging area. For example, a scan is performed as helical scan.

Next, as shown in FIG. 5, a tomographic image is reconstructed from the projection data (S21).

The image reconstruction unit 303 will reconstruct a tomographic image of a cross-sectional plane of the imaging area of the subject based on the projection data obtained by the performed scan.

More specifically, the projection data acquired by the data acquisition system 24 may be applied with preprocessing such as the offset correction, logarithm correction, X-ray dose correction, sensitivity correction, then filtering may be applied to the preprocessed projection data may be. The filtering may include a Fourier Transform, then a convolution of image reconstruction function, and finally an inverted Fourier Transform. Thereafter, the three-dimensional back projection may be applied to the thus filtered projection data, postprocessing may then be applied to reconstruct a tomographic image. In the preferred embodiment the image reconstruction unit 303 calculates a CT value for each pixel from the projection data obtained by performing a scan to reconstruct a tomographic image.

Next, as shown in FIG. 5, the tomographic image is used to calculate the scattered radiation beam data (S31).

For the X-ray emitted from the X-ray tube 21 to the imaging area of the subject when performing a scan of the subject, the scattered radiation beam data calculation unit 304 estimates the scattered radiation beam which is scattered by the imaging area of the subject to compute the scattered radiation beam data.

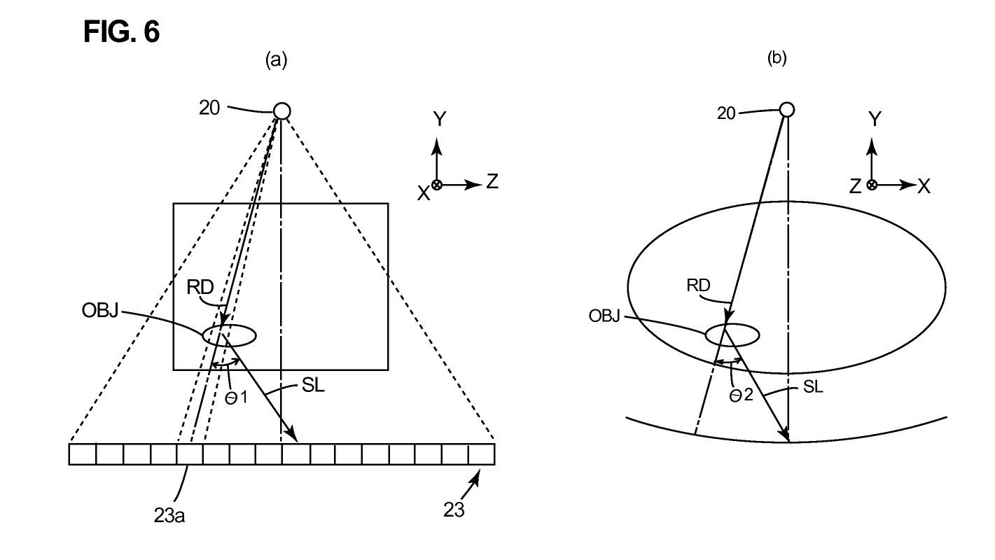

Now referring to FIG. 6, there is shown a side view illustrating the behavior of the scattered radiation beam scattered by the imaging area of the subject when performing a scan on the imaging area of the subject by the scanning gantry 2, in the first preferred embodiment of the present invention. In FIG. 6, FIG. 6 (a) shows y-z plane and FIG. 6 (b) shows x-y plane.

As shown in FIG. 6 (a) and FIG. 6 (b), for the X-ray emitted to the imaging area of the subject in accordance with the scan condition set by the scan condition setting unit 302, the scattered radiation beam data calculation unit 304 estimates the scattered radiation beam SL, which is scattered by the imaging area of the subject in the scattering direction which is different from the radiation direction RD in which the X-ray tube 21 emits radiation beam to each of the detecting elements 23a of the X-ray detector 23, thereby to compute the scattered radiation beam data. In the imaging area of the subject, between the object OBJ part and any part except for the object OBJ part, both having a different density each other, scattering angles θ1, θ2 with respect to the radiation direction RD are different from each other, the scattered radiation beam data is to be computed considering each of these different densities.

In the first preferred embodiment, the density distribution calculation unit 341 of the scattered radiation beam data calculation unit 304 calculates the density distribution of the imaging area of the subject based on the tomographic image reconstructed by the image reconstruction unit 303. Here it calculates the density distribution of the imaging area of the subject from the CT value of each pixel in the tomographic image. Then, based on the density distribution calculated by the density distribution calculation unit 341, the scattered radiation beam data calculation unit 304 calculates the scattered radiation beam data. In the first preferred embodiment the scattered radiation beam data calculation unit 304 estimates by computation of the scattered radiation beam corresponding to the scan condition set by the scan condition setting unit 302 and to the density distribution calculated by the density distribution calculation unit 341 from the scattered radiation beam characteristics information stored in the storage unit 61, thereby to compute the scattered radiation beam data.

For example, the scattered radiation beam data included in the projection data can be determined by using the scattered radiation beam characteristics information stored as a lookup table which corresponds to the characteristics of scattered radiation beam that the X-ray emitted from the X-ray tube 21 scatters at the subject because of the phenomenon such as photoelectric effect, Rayleigh scattering, and Compton scattering at the time of scanning, with the energy distribution of X-ray transmitted as well as with the transmission length of X-ray which transmits through the subject.

More specifically, the scattered radiation beam data may be determined as shown in the following equations (1), (2), (3), (4), and (5).

Pphθ(D,E,z,θ)=Pphcs(D,E)*Pphdc(D,E,z,θ) (1)

Preθ(D,E,z,θ)=Precs(D,E)*Predc(D,E,z,θ) (2)

Pcomθ(D,E,z,θ)=Pcomcs(D,E)*Pcomdc(D,E,z,θ) (3)

Pcolθ(D,E,z,θ)=Pph(D,E,z,θ)+Pre(D,E,z,θ)+Pcom(D,E,z,θ) (4)

Rdis(x,y,view,θ)=L(x,y,view)*tan θ/detector_colimation (5)

In the above equations, D means the abbreviation of D (x, y), which designates to the density distribution in the imaging area of the subject corresponding to the tomographic image I (x, y), in the x-y plane where the vertical line is in the body axis direction z, and in which a plurality of pixels are arranged in the x direction and y direction. E means the abbreviation of E (x, y, view), which indicates the energy distribution of X-ray emitted in each view around the subject at the time of scanning, in correspondence with the tomographic image I (x, y). L (x, y, view) indicates the distance from the imaging area of the subject to the X-ray detector 23 in correspondence with a given pixel pix (x, y) in the tomographic image I (x, y). Pph θ (D, E, z, θ) designates to the scattering probability in the θ direction. Pphcs (D, E) designates to the scattering probability of photoelectric effect. Pphdc (D, E, z, θ) indicates the probability of scattering in the direction θ when the photoelectric effect occurs. In other words Pph θ is derived from Pphcs and Pphdc. Pre θ (D, E, z, θ) is the scattering probability in the θ direction in Rayleigh scattering. Precs (D, E) is the scattering probability of Rayleigh scattering. Predc (D, E, z, θ) is the probability of scattering in the direction θ when Rayleigh scattering occurs. In other words Pre θ can be derived from Precs and Predc. Pcom θ (D, E, z, θ) is the scattering probability in the direction θ in Compton scattering. Pcomcs (D, E) is the dispersion probability of Compton scattering. Pcomdc (D, E, z, θ) is the probability of scattering in the direction θ when Compton scattering occurs.

In the first preferred embodiment, based on the tomographic image reconstructed, the density distribution of the imaging area of the subject in correspondence with the tomographic image is determined, and then based on the density distribution, the probability of occurrence of scattering is derived on the tomographic image to determine the scattered radiation beam data.

Next, as shown in FIG. 5, the scattered radiation beam data is used for the scattered radiation beam correction for the projection data (S41).

In the first preferred embodiment, after the image reconstruction unit 303 receives once again the projection data obtained by performing a scan from the storage unit 61, the image reconstruction unit 303 performs the scattered radiation beam correction using the scattered radiation beam data calculated by the scattered radiation beam data calculation unit 304.

In the first preferred embodiment, the scattered radiation beam data calculated by the scattered radiation beam data calculation unit 304 is used to calculate the correction coefficient coefA (row, ch, proj), and coefB (i, ch, proj) for the correction of projection data.

More specifically, as shown in the following equations (6) and (7), the correction coefficients coefA (row, ch, proj) and coefB (i, ch, proj) may be calculated. In the equations ‘row’ designates to the position of detector elements arranged in the row direction j in the X-ray detector 23. The term ‘ch’ designates to the position of the detector elements 23a arranged in the channel direction i in the X-ray detector 23. The term ‘proj’ designates to the projection data to be compensated for. G is a function to calculate the amount of Ray to be input in row, ch from the Geometry in case of θ, i, j. Ps θ is the value indicated by Ps θ=Pre θ+Pcom θ. R (i, j, view, p, e) designates to the amount of X-ray (or number of photons), which may have energy e at the projection of the amount P. R (i, j, view, p, e) can be indicated by R (i, j, view, p, e)=R (p)−Pcol θ*R (p).

$$\begin{matrix}CoefB ( row , ch , view ) = {\sum }_{0} {\sum }_{i}^{Row} {\sum }_{j}^{ch} {\sum }_{p}^{proj} {\sum }_{e}^{Energy} G ( θ , i , j , view ) \cdot Ps θ ( i , j , view , p , e ) \cdot R ( i , j , view , p , e ) & ( 6 ) \\ CoefA = \frac{10}{1 - \sum coefB} & ( 7 )\end{matrix}$$

Then, based on the following equation (8), the corrected projection data Proj′ (row, ch) may be calculated. In the preferred embodiment it is assumed that collimators (not shown in the figure) are provided for preventing scattered X-ray from penetrating into the detecting elements 23a in the channel direction i of the X-ray detector 23. However, if the collimators are not provided then it may be preferable that similar correction is equally applied to the channel direction i.

$${Proj}^{′} ( row , ch ) = CoefA ( row , ch , proj ) \times Proj ( row , ch ) - {\sum }_{i = start\_row ( row )}^{and\_row ( row )} CoefB ( i , ch , proj ) \times Proj ( i , ch ) \qquad ( 8 )$$

Next, as shown in FIG. 5, a scattered radiation beam compensated image is reconstructed based on the corrected projection data (S51).

In the present embodiment the image reconstruction unit 303 reconstructs the scattered radiation corrected image that is scattered radiation beam corrected based on the corrected projection data as stated above.

More specifically, after the corrected projection data is applied with the preprocessing including such as the offset correction, logarithm correction, X-ray dose correction, and sensitivity correction, the filtering is applied to thus preprocessed projection data. In this example the data is applied with the Fourier Transform, the convolution of an image reconstruction function, and then the invert Fourier Transform. Then, the projection data thus filtered is applied with a three-dimensional back projection processing, and the postprocessing thereafter. In this manner the scattered radiation corrected image is reconstructed.

Next, as shown in FIG. 5, the scattered radiation corrected image may be displayed (S61).

In the present embodiment, the scattered radiation corrected image, reconstructed by the image reconstruction unit 303, may be displayed on the display screen by the display device 51.

As can be appreciated from the foregoing description, in the first preferred embodiment, from the projection data obtained by performing a scan on the imaging area of the subject by the scanning gantry 2 so as to accommodate with the scan condition set by the scan condition setting unit 302, the image reconstruction unit 303 reconstructs a tomographic image for a cross-sectional plane of the imaging area of the subject. In the scattered radiation beam data calculation unit 304, based on the tomographic image reconstructed by the image reconstruction unit 303, a density distribution calculation unit 301a calculates the density distribution in the imaging area of the subject. Thereafter, based on the density distribution calculated by the density distribution calculation unit 341, the scattered radiation beam data calculation unit 304 calculates the scattered radiation beam data. Then the image reconstruction unit 303 performs the scattered radiation correction of the projection data obtained by the scanning gantry 2 performing a scan in accordance with the scan condition set by the scan condition setting unit 302, by means of the scattered radiation beam data calculated by the scattered radiation beam data calculation unit 304. Then based on the projection data thus scattered radiation corrected, the image reconstruction unit 303 reconstructs the scattered radiation corrected image. As can be seen from the foregoing description, in the present preferred embodiment, the density distribution is derived from the tomographic image of the imaging area of the subject to calculate the scattered radiation beam data, then the scattered radiation beam data is used for the scattered radiation correction of the projection data, then the scattered radiation corrected image is reconstructed from the scattered radiation corrected projection data. By doing this, the present embodiment considers the difference of behavior of the scattered radiation beam in accordance with the object within the imaging area of the subject to perform the scattered radiation correction for eliminating the scattered radiation beam data causing noises in the projection data, so that the artifacts may be prevented from developing in the tomographic image reconstructed by using the projection data, allowing improvement of image quality.

### Second Embodiment

From now on a second preferred embodiment of the present invention may be described in greater detail.

In the X-ray CT apparatus 1 of the second preferred embodiment, the arrangement and operation of the central processing unit 30 is different from that shown in the first preferred embodiment. Except for this the present preferred embodiment is similar to the first embodiment. The description of members already described in the foregoing description may be omitted in the following.

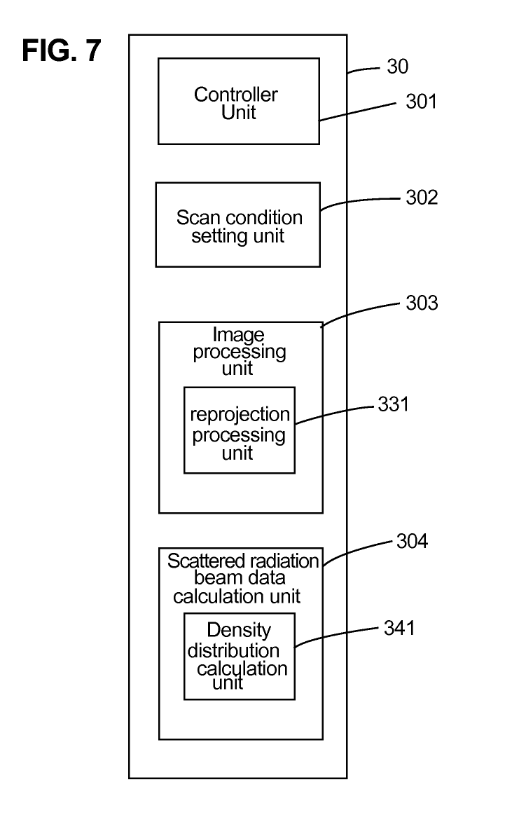

Now referring to FIG. 7, there is a schematic block diagram illustrating the arrangement of the central processing unit 30 in the second preferred embodiment in accordance with the present invention.

As shown in FIG. 7, in the central processing unit 30 in accordance with the present embodiment, the image reconstruction unit 303 includes a reprojection processing unit 331.

In the image reconstruction unit 303, the reprojection processing unit 331 performs reprojection processing of the tomographic image previously reconstructed by the image reconstruction unit 303 to obtain the reprojection data. The image reconstruction unit 303 then performs the scattering X-ray correction processing of the reprojection data obtained by the reprojection processing unit 331, using the scattered radiation beam data calculated by the scattered radiation beam data calculation unit 304, then reconstructs the scattered radiation corrected image based on the reprojection data that has been scattered radiation corrected for.

Now the operation of the X-ray CT apparatus 1 of the second preferred embodiment may be described in greater detail herein below.

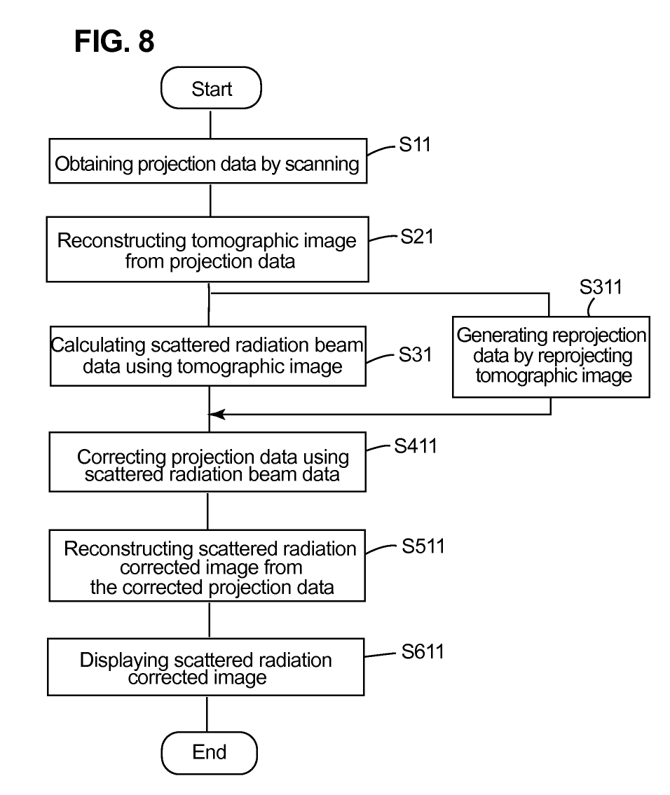

Now referring to FIG. 8, there is shown a schematic flow diagram illustrating the operation of the X-ray CT apparatus 1 in accordance with the second preferred embodiment of the present invention.

First, as shown in FIG. 8, in a manner similar to the first preferred embodiment, a scan is conducted to obtain projection data (S11), then a tomographic image is reconstructed based on the projection data (S21).

Then, as shown in FIG. 8, in a manner similar to the first preferred embodiment, the tomographic image is used to calculate the scattered radiation data (S31).

At this point, as shown in FIG. 8, the tomographic image is reprojected in order to generate reprojection data (S311).

In this second preferred embodiment, the tomographic image reconstructed by the image reconstruction unit 303 as has been stated above is further reprojected by the reprojection processing unit 331 to obtain the reprojection data. More specifically, the reprojection processing unit 331 will perform reprojection such that projection from a plurality of directions along with the cross-sectional plane of the tomographic image is performed, from the circumference of the tomographic image reconstructed by the image reconstruction unit 303 based on the projection data obtained in accordance with the scan condition set by the scan condition setting unit 302, thereby to generate the reprojection data.

Next, as shown in FIG. 8, the scattered radiation data may be used to compensate the reprojection data for the scattered radiation beam (S411).

In this second preferred embodiment, the scattered radiation beam data calculated by the scattered radiation beam data calculation unit 304 is used to correct thus obtained reprojection data for the scattered radiation beam by the image reconstruction unit 303.

In this second preferred embodiment, as similar to the first preferred embodiment, the scattered radiation beam data calculated by the scattered radiation beam data calculation unit 304 may be used to calculate the correction coefficients CoefA (row, ch, reproj), and CoefB (i, ch, reproj) for correcting the reprojection data.

Then, the projection data after compensation Reproj′ (row, ch) may be calculated based on the following equation (9).

$${Reproj}^{′} ( row , ch ) = coefA ( row , ch , reproj ) \times Reproj ( row , ch ) - {\sum }_{i = start\_row ( row )}^{and\_row ( row )} coefB ( i , ch , reproj ) \times Reproj ( i , ch ) \qquad ( 9 )$$

Next, as shown in FIG. 8, based on the corrected projection data, a scattered radiation corrected image may be reconstructed (S511).

In this second preferred embodiment, from the reprojection data that has been corrected as stated above, the image reconstruction unit 303 reconstructs the scattered corrected image that has scattered radiation correction processed.

Next, as shown in FIG. 8, the scattered radiation corrected image may be displayed (S611).

In this second preferred embodiment, the scattered radiation corrected image reconstructed by the image reconstruction unit 303, may be displayed on the display screen by the display device 51.

As can be appreciated from the foregoing description, in the second preferred embodiment, a previously generated tomographic image is reprojected to generate reprojection data, then in a manner similar to the preceding first preferred embodiment, the scattered radiation beam data calculated by the scattered radiation beam data calculation unit 304 is used to scattered radiation correct the reprojection data by the image reconstruction unit 303. Thereafter, based on the reprojection data that has scattered radiation corrected, the image reconstruction unit 303 reconstructs a scattered radiation corrected image. By doing this, in this second preferred embodiment, by taking into account the difference of behavior of the scattered radiation beam depending on the presence of object in the imaging area of the subject, scattered radiation correction is performed to eliminate scattered radiation beam data, which causes noise, from the reprojection data, the artifacts may be prevented from occurring in the tomographic image reconstructed by using such reprojection data, allowing improvement of image quality.

### Third Embodiment

A third preferred embodiment in accordance with the present invention will be described in greater detail herein below.

In the X-ray CT apparatus 1 of the preferred third embodiment the operation of the central processing unit 30 is different from that of the preceding first preferred embodiment. Except for this the third preferred embodiment is similar to the first embodiment. Therefore the description of similar member may be omitted.

The operation of the X-ray CT apparatus 1 in accordance with the third preferred embodiment may be described in greater detail herein below.

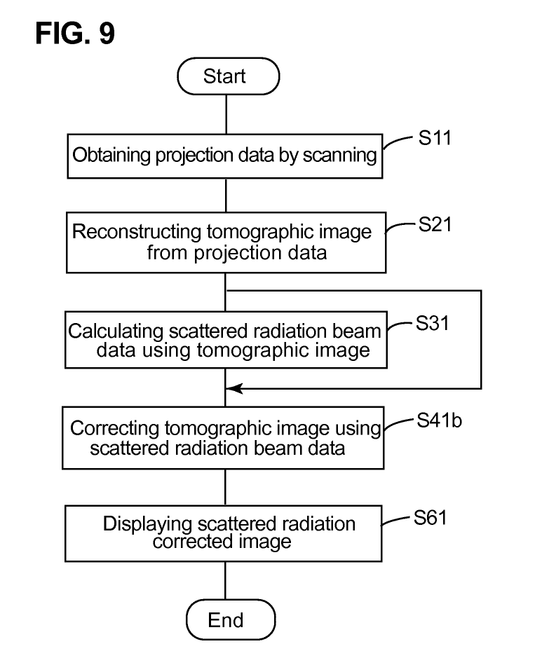

Now referring to FIG. 9, there is shown a schematic flow diagram illustrating the operation of the X-ray CT apparatus 1 in accordance with the third preferred embodiment of the present invention.

First, as shown in FIG. 9, in a manner similar to the preceding first preferred embodiment, a scan is conducted to obtain the projection data (S11), and then based on the projection data a tomographic image is reconstructed (S21).

Then, as shown in FIG. 9, in a manner similar to the preceding first preferred embodiment, the tomographic image is used to calculate the scattered radiation beam data (S31).

Next, as shown in FIG. 9, the scattered radiation beam data is used to perform the scattered radiation correction of the tomographic image (S41).

In this embodiment, using the scattered radiation beam data calculated by the scattered radiation beam data calculation unit 304, the image reconstruction unit 303 performs the scattered radiation correction processing of the tomographic image thus obtained as stated above.

More specifically, the correction is to be conducted as shown in the following equation (10).

$${I}^{′} ( x , y , image ) = coefA ( x , y , image ) \cdot I ( x , y , image ) - {\sum }_{k}^{image} {\sum }_{v}^{view} {\sum }_{j}^{y} {\sum }_{i}^{x} coefB ( i , j , k , v ) - I ( i , j , k , v ) \qquad ( 10 )$$

Next, as shown in FIG. 9, the scattered radiation corrected image may be displayed (S61).

In this third preferred embodiment, the scattered radiation corrected image that has scattered radiation corrected from the tomographic image by the image reconstruction unit 303, may be displayed on the display screen by the display device 51.

As can be appreciated from the foregoing description, in the third preferred embodiment, by using the scattered radiation beam data calculated by the scattered radiation beam data calculation unit 304 in a manner similar to the preceding first preferred embodiment, the tomographic image previously reconstructed by the image reconstruction unit 303 is applied with the scattered radiation correction to obtain scattered radiation corrected image. By doing this, in the third preferred embodiment, by taking into account the difference of behavior of the scattered radiation beam depending on the presence of object in the imaging area of the subject, the scattered radiation correction is performed to eliminate noises caused by the scattered radiation m data from the tomographic image so that the artifacts may be prevented from developing, allowing improvement of the image quality.

In the third preferred embodiment as has been described above, the X-ray CT apparatus 1 corresponds to the radiation imaging apparatus in accordance with the present invention. The scanning gantry 2 corresponds to the scan unit in accordance with the present invention. Also in the third preferred embodiment as has been described above, the X-ray tube 20 corresponds to the radiation section in accordance with the present invention. In the preferred embodiment as has been described above, the X-ray detector 23 corresponds to the detecting section in accordance with the present invention. In the third preferred embodiment as has been described above, the revolving unit 27 corresponds to the revolving section in accordance with the present invention. Further, in the preferred embodiment as has been described above, the display device 51 corresponds to the display unit in accordance with the present invention. In the third preferred embodiment as has been described above, the storage unit 61 corresponds to the scattered radiation beam characteristics information storage unit in accordance with the present invention. Furthermore, in the preferred embodiment as has been described above, the scan condition setting unit 302 corresponds to the scan condition setting unit in accordance with the present invention. In the preferred embodiment as has been described above, the image reconstruction unit 303 corresponds to the image reconstruction unit in accordance with the present invention. In the third preferred embodiment as has been described above, the scattered radiation beam data calculation unit 304 corresponds to the scattered radiation beam data calculation unit in accordance with the present invention. In the third preferred embodiment as has been described above, the reprojection processing unit 331 corresponds to the reprojection processing section in accordance with the present invention. In addition, in the third preferred embodiment as has been described above, the density distribution calculation unit 341 corresponds to the density distribution calculation section in accordance with the present invention.

It is to be noted here that the practice of the present invention is not limited to the preferred embodiments as have been described above, rather various modifications and changes may be made.

For example, in the preferred embodiments as have been described above, although examples using X-ray as the radiation beam have been described, the present invention is not limited thereto. For example, other radiation such as Gamma radiation can be used equally.

1. A radiation imaging apparatus comprising:
a scan unit including a radiation section configured to emit a radiation beam and a detecting section having a plurality of detecting elements arranged to detect the radiation beam emitted from said radiation section, wherein the scan unit is configured to perform a scan in which said radiation section emits the radiation beam to an imaging area of a subject, and said detecting section detects the radiation beam transmitted through said imaging area to obtain projection data of said imaging area;
a scan condition setting unit configured to set a scan condition for the scan performed by said scan unit;
a scattered radiation beam data calculation unit, configured to calculate scattered radiation beam data, in the radiation beam emitted from said radiation section to the imaging area of the subject in accordance with the scan condition set by said scan condition setting unit wherein the scattered radiation beam data is calculated by estimating a scattered radiation beam scattered in a scattering direction which is different from a radiation direction in which a radiation beam is emitted from said radiation section to each of said detecting elements in said detecting section; and
an image reconstruction unit configured to reconstruct a scattered radiation corrected image to which scattered radiation correction processing is applied with respect to a cross-sectional plane in the imaging area of the subject, using the projection data obtained from a scan performed by said scan unit in accordance with the scan condition set by said scan condition setting unit, and the scattered radiation beam data calculated by said scattered radiation beam data calculation unit,
wherein:
said image reconstruction unit is configured to reconstruct a tomographic image with respect to a cross-sectional plane of the imaging area based on said projection data; and
said scattered radiation beam data calculation unit includes a density distribution calculation section configured to calculate a density distribution of the imaging area based on the tomographic image reconstructed by said image reconstruction unit, and to calculate the scattered radiation beam data based on the density distribution calculated by said density distribution calculation section.

2. A radiation imaging apparatus according to claim 1, wherein said image reconstruction unit is configured to apply the scattered radiation processing to the projection data, and thereafter, to reconstruct said scattered radiation corrected image based on the projection data to which said scattered radiation correction processing has been applied.

3. A radiation imaging apparatus according to claim 1, wherein said image reconstruction unit includes a reprojection processing section for configured to obtain reprojection data by applying reprojection processing to said reconstructed tomographic image, and after applying the scattered radiation correction processing to the reprojection data obtained by said reprojection section, is configured to reconstruct said scattered radiation corrected image based on the reprojection data to which said scattered radiation correction processing has been applied.

4. A radiation imaging apparatus according to claim 1, wherein said image reconstruction unit is configured to obtain said scattered radiation corrected image by applying the scattered radiation correction processing to said reconstructed tomographic image, using said scattered radiation beam data.

5. A radiation imaging apparatus according to claim 1, further comprising a scattered radiation beam characteristics information storage unit configured to store scattered radiation beam characteristics information in which characteristics of said scattered radiation beam are associated with said scan condition and density of the imaging area of the subject,
wherein said scattered radiation beam data calculation unit is configured to calculate the scattered radiation beam data by estimating said scattered radiation beam corresponding to said scan condition and said density distribution, based on the scattered radiation beam characteristics information stored in said scattered radiation beam characteristics information storage unit.

6. A radiation imaging apparatus according to claim 1, wherein said scan unit includes a revolving section configured to revolve said radiation section and said detecting section around the subject, and configured to perform a scan by allowing said revolving section to revolve said radiation section and said detecting section around the subject for emitting a radiation beam to the imaging area of the subject from periphery of the imaging area of the subject and detecting the radiation beam transmitted through the imaging area.

7. A radiation imaging apparatus according to claim 6, wherein:
said radiation section is configured to emit the radiation beam so as to be expanded radially into a revolving direction in which said radiation section is revolved by said revolving section and into a revolving axis direction; and
said detecting section has a plurality of said detecting elements arranged so as to correspond to said revolving direction and said revolving axis direction.

8. A radiation imaging apparatus according to claim 1, further comprising a display unit configured to display said scattered radiation corrected image on a display screen.

9. A radiation imaging apparatus according to claim 1, wherein said radiation section is configured to emit an X-ray as said radiation beam.

10. A radiation imaging method for allowing a scan unit to perform a scan in accordance with a scan condition, wherein the scan unit includes a radiation section configured to emit a radiation beam to an imaging area of a subject and includes a detecting section having a plurality of detecting elements arranged to detect the radiation beam through the imaging area to obtain projection data of the imaging area, the method comprising:
a scattered radiation beam data calculating step of calculating scattered radiation beam data, by estimating a scattered radiation beam scattered in a scattering direction which is different from a radiation direction in which a radiation beam is emitted from said radiation section to each of detecting elements in said detecting section, in radiation beam emitted from said radiation section to said imaging area in accordance with said scan condition; and
a scattered radiation corrected image reconstructing step of reconstructing a scattered radiation corrected image to which scattered radiation correction processing is applied with respect to a cross-sectional plane in said imaging area, by using the projection data obtained from a scan performed by said scan unit, and the scattered radiation beam data calculated in said scattered radiation beam data calculating step;
wherein:
said scattered radiation beam data calculating step comprises
a tomographic image reconstructing step of reconstructing a tomographic image with respect to a cross-sectional plane of the imaging area based on the projection data;
a density distribution calculating step of calculating a density distribution of said imaging area based on the tomographic image reconstructed in said tomographic image reconstructing step; and
a data calculating step for calculating the scattered radiation beam data based on the density distribution calculated in said density distribution calculating step.

11. A radiation imaging method according to claim 10, wherein
said scattered radiation corrected image restructuring step comprises the steps of
applying a scattered radiation correction processing to the projection data based on said scattered radiation beam data; and
reconstructing said scattered radiation corrected image based on the projection data to which said scattered radiation correction processing has been applied.

12. A radiation imaging method according to claim 10, wherein
said scattered radiation corrected image reconstructing step comprises the steps of reprojecting the tomographic image to obtain reprojection data;
applying a scattered radiation correction processing to the reprojection data based on the scattered radiation beam data; and
reconstructing said scattered radiation corrected image based on the reprojection data to which said scattered radiation correction processing has been applied.

13. A radiation imaging method according to claim 10, wherein said scattered radiation corrected image reconstructing step comprises the step of
applying scattered radiation correction processing to the tomographic image based on the scattered radiation data, to reconstruct said scattered radiation corrected image.

14. A radiation imaging method according to claim 10, wherein
said scattered radiation beam data calculating step comprises the step of
calculating said scattered radiation beam data by estimating the scattered radiation beam corresponding to said scan condition and density distribution, based on scattered radiation beam characteristics information in which scattered radiation beam are associated with said scan condition and density of said imaging area.

15. A radiation imaging method according to claim 10, further comprising performing the scan by revolving said radiation section and said detecting section around the subject for emitting a radiation beam to said imaging area from the periphery of said imaging area and detecting the radiation beam transmitted through said imaging area.

16. A radiation imaging method according to claim 10, further comprising
a displaying step of displaying said scattered radiation corrected image.
